(19) United States
(12) Patent Application Publication
(10) Pub. No.: US 2017/0312456 A1
(43) Pub. Date: Nov. 2, 2017

(54) Skin Desensitizing Device

(71) Applicant: David Bruce Phillips, Waterford, VA (US)

(72) Inventor: David Bruce Phillips, Waterford, VA (US)

(21) Appl. No.: 15/499,886

(22) Filed: Apr. 27, 2017

Related U.S. Application Data
(60) Provisional application No. 62/328,085, filed on Apr. 27, 2016.

(51) Int. Cl. A61M 5/42; A61H 23/02; A61N 1/04; A61N 1/36

(52) U.S. Cl. CPC A61M 5/422; A61N 1/36021; A61H 23/02; A61N 1/0456; A61M 2205/3372; A61M 2205/3306; A61M 2205/3303; A61M 2205/50; A61M 2205/8206; A61M 2210/04; A61M 2205/581

(57) ABSTRACT

A skin desensitizing device for temporarily numbing and/or cooling a surface of a subject's skin. The skin desensitizing device comprises a controller, a skin-bracing interface, and at least one overloading-neural source, which includes (i) a transcutaneous electrical nerve stimulator (TENS)-like device capable of electrically stimulating superficial skin neurons of the subject's skin into a state of fibrillation, (ii) a thermoelectric device capable of cooling the subject's skin to approximately 30° F. to 38° F., and (iii) a vibration device capable of vibrating the subject's skin to overload the skin's vibration sensors.

The current application claims a priority to the U.S. Provisional Patent application Ser. No. 62/628,085 filed on Apr. 27, 2016.

### FIELD OF THE INVENTION

The present invention generally relates to an apparatus that temporarily numbs a patient's skin to allow for more painless performance of various medical procedures, including those involving needle sticks.

### BACKGROUND OF THE INVENTION

During routine medical procedures that involve minor temporary trauma such as needle sticks, laser ablation or electro cauterization and the like, including, for example, venipuncture, skin pricks, diabetic glucose testing, shots, vaccines, arthritis treatments, laser procedures (e.g. hair or tattoo removal), and minor outpatient surgeries (e.g. skin tag removal, hemorrhoid treatment, etc.), patients often become uncomfortable and/or nervous about the use of the needle. Patients also often experience pain with the needle stick. However, in current medical practice, the patient is left to experience this pain and/or anxiety without any assistance to ease these symptoms. Patients are generally not provided satisfactory aid to numb the skin or the area to be subjected to the needle stick. With respect to certain patients, such as pediatric patients, high anxiety and susceptibility to pain can make these medical procedures mentally stressing and unbearable.

Previous numbing aids and devices have attempted to numb a target area of a patient's skin surface that is to be acted upon by a medical treatment device (e.g. a syringe, needle, lance, or ablation laser). Some aids that have attempted to numb the skin include, for example, ethyl chloride spray and/or numbing devices. Previous devices have incorporated a Peltier effect element to cool the area of the skin and/or a vibration means to stimulate the same area of the skin. However, such devices are often incorporated into a housing that includes the syringe or needle, which requires the device to be operated by a doctor and/or nurse, and to remain on the skin during the needle stick. Further, such devices do not take away the apprehension of the patient towards the use of the needle.

Alternative devices that have attempted to numb an injection site of a patient's skin for a pain-free injection have incorporated transcutaneous electrical nerve stimulator (TENS) machines. TENS machines deliver small electrical pulses to the body via electrodes placed on the skin. By delivering an electric current to the body through the skin to the nerves under the skin, the electric current can be manipulated and delivered in a manner to reduce the ability of the nerves to sense pain. However, such devices often require the electrodes to remain on the skin during the needle stick, which can cause an allergic reaction in some patients. These devices also require the precise placement of the electrodes onto the patient's skin and the operation of the device by a doctor and/or nurse.

Other devices have attempted to achieve a numbing effect by producing a chemical reaction on the skin's surface that numbs the skin. For example, one device consists of two sealed chambers containing ammonium nitrate and water, respectively, that are brought together to react to produce a rapid endothermic reaction that can be used to numb a subject's skin.

Accordingly, there is a need to provide a device that would rapidly and temporarily numb the superficial skin neurons of a patient to allow for various medical procedures involving needle sticks to be performed painlessly and without mental distress to the patient. Moreover, a need exists to provide such a device that can be easily operated by the patient and/or clinician.

### SUMMARY OF THE INVENTION

It is an object of the present invention to provide a device that temporarily numbs the superficial skin neurons of a patient prior to a medical procedure involving minor temporary trauma such as needle sticks, laser ablation or electro cauterization and the like.

It is a further object of the present invention to provide such a numbing device that does not (i) have to remain on the patient's skin during the needle stick, (ii) require the use of electrodes, and (iii) have to be operated by a doctor, nurse, or clinician.

According to one aspect, a skin desensitizing device is provided for temporarily numbing and/or cooling a surface of a subject's skin. The device includes a controller and at least two of (i) a transcutaneous electrical nerve stimulator (TENS)-like device capable of electrically stimulating superficial skin neurons of the subject's skin into a state of fibrillation, (ii) a thermoelectric device capable of cooling the subject's skin to approximately 30° F. to 38° F., and (iii) a vibration device capable of vibrating the subject's skin to overload the skin's vibration sensors.

According to another aspect, a method is provided of temporarily numbing and/or cooling a surface of a subject's skin. The method includes at least two of the following steps: (a) cooling the subject's skin to approximately 30° F. to 38° F., (b) electrically stimulating superficial skin neurons of the subject's skin into a state of fibrillation, and (c) vibrating the subject's skin to overload the skin's vibration sensors. A Peltier-effect, thermoelectric device can perform the step of cooling the subject's skin. A transcutaneous electrical nerve stimulator (TENS)-like device can perform the step of electrically stimulating the superficial skin neurons into a state of fibrillation. A piezoelectric vibration device can perform the step of vibrating the subject's skin.

According to yet another aspect, a method of performing a medical procedure on a subject involving a needle stick is provided. The method includes temporarily numbing and/or cooling a surface of the subject's skin that is be punctured by a needle for the needle stick, and, subsequent to the step of temporarily numbing and/or cooling the surface of the subject's skin, performing the medical procedure involving the needle stick. The step of temporarily numbing and/or cooling the surface of the subject's skin comprises at least two of the following steps: (i) cooling the subject's skin to approximately 30° F. to 38° F., (ii) electrically stimulating superficial skin neurons of the subject's skin into a state of fibrillation, and (iii) vibrating the subject's skin to overload the skin's vibration sensors. A Peltier-effect, thermoelectric device can perform the step of cooling the subject's skin. A transcutaneous electrical nerve stimulator (TENS)-like device can perform the step of electrically stimulating the superficial skin neurons into a state of fibrillation. A piezoelectric vibration device can perform the step of vibrating the subject's skin.

Other aspects and advantages of the present invention will become apparent upon consideration of the following detailed description.

### BRIEF DESCRIPTION OF THE DRAWINGS

FIG. 1 illustrates a skin cooling device being used by a patient according to an embodiment of the invention.

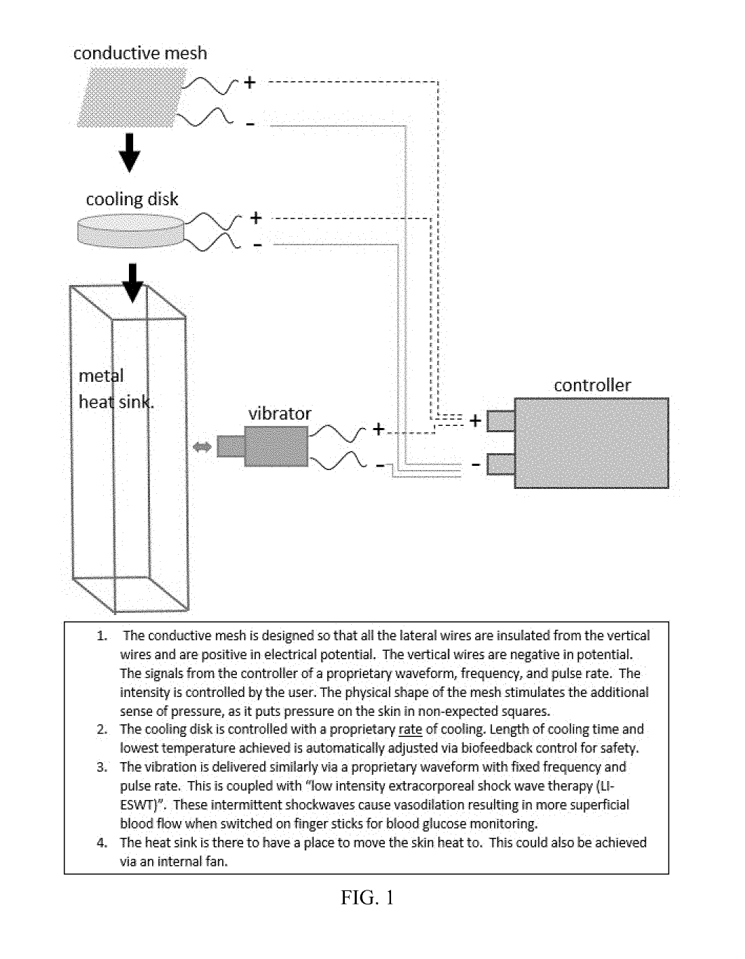

FIG. 2 is a diagram of a skin cooling device according to an embodiment of the invention.

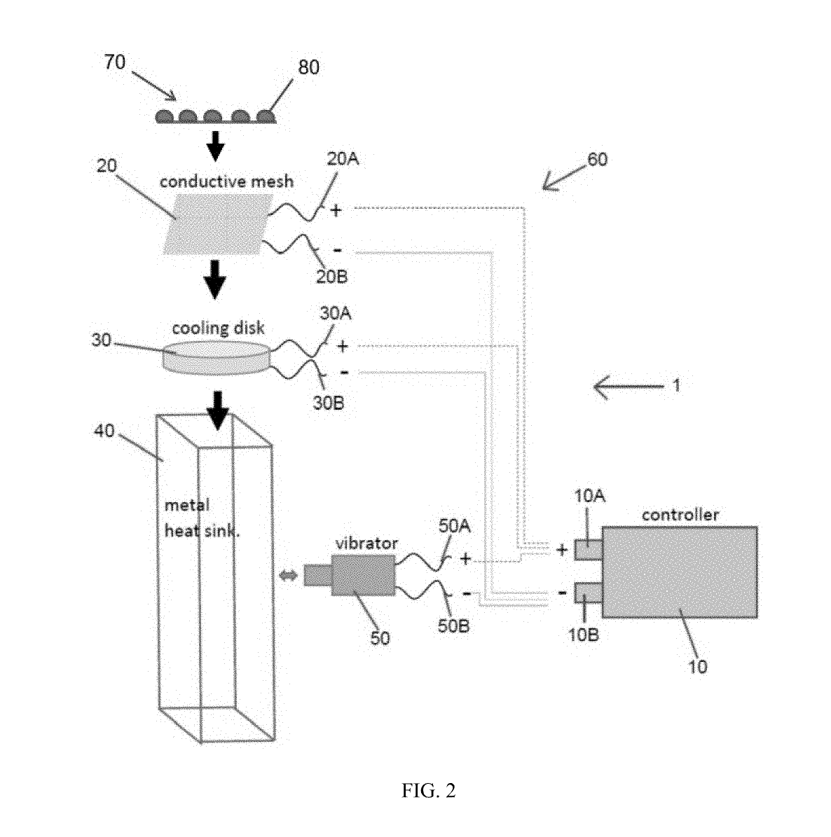

### DETAILED DESCRIPTION OF THE INVENTION

All illustrations of the drawings are for the purpose of describing selected versions of the present invention and are not intended to limit the scope of the present invention.

The present invention is a skin desensitizing device, which generally relates to an apparatus that temporarily numbs and/or cools a patient's skin to allow for painless performance of various medical procedures involving needle sticks. More specifically, the present invention relates to a handheld device that incorporates at least one of three separate sources of neural overload to temporarily cool and/or numb the superficial skin neurons of a target area of the patient's skin. The three separate sources of neural overload include at least one of following actions: (i) electrically stimulating the superficial skin neurons into a state of fibrillation that thus, blocks subsequent signals; (ii) cooling the skin to approximately 30° F. to 38° F. to overload the skin's sense of hot, cold, or touch; and (iii) vibrating the skin to overload the skin's vibration sensors.

The present invention comprises a controller 1, at least one neural-overloading source 2, a skin-bracing interface 3, and a power source 4. As described before, the at least one neural-overloading source 2 is used to temporarily cool and/or numb the superficial skin neurons of a target area of the patient's skin. The controller 1 is used to manage and dictate the actions of the at least one neural-overloading source 2. The skin-bracing interface 3 is the portion of the present invention that physically contacts the patient's skin while using the at least one neural-overloading source 2. The power source 4 is used to provide electrical power to the at least one neural-overloading source 2, the controller 1, and/or any other necessary component. The power source 4 is preferably a disposable or rechargeable battery because the present invention is designed to be a handheld device. However, an external power source 4, such as an electric outlet, could also be used to provide electrical power to the components of the present invention.

The configuration of the aforementioned components allows the present invention effectively and efficiently desensitize a target area of the patient's skin. The at least one neural-overloading source 2 can be, but is not limited to, a transcutaneous electrical nerve stimulator (TENS)-like device, a thermoelectric device, a vibration device, or any combination thereof. The TENS-like device allows the present invention to electrically stimulate the superficial skin neurons into a state of fibrillation. The thermoelectric device allows the present invention to cool the patient's skin to approximately 30° F. to 38° F., which overloads the patient skin's sense of hot, cold, or touch. The vibration device allows the present invention to vibrate the patient's skin, which overloads the skin's vibration-sensing ability. Moreover, the at least one neural-overloading source 2 is electrically connected to the power source 4 through the controller 1, which allows the controller 1 to manage how electrical power is delivered to each neural-overloading source 2. The skin-bracing interface 3 is operatively coupled to the at least one neural-overloading source 2 so that the skin-bracing interface 3 can be used as a medium between the patient's skin and the at least one neural-overloading source 2.

In general, the present invention is a handheld device and is configured to be placed or touched against the patient's skin in order to apply the cooling and/or numbing effect onto the patient's skin. The present invention is preferably an automatic “touch-and-go” device, meaning, the present invention is simply placed against the patient's skin for up to ten seconds, and the patient's skin will thereafter be sufficiently numbed for at least thirty seconds. This rapid numbing of the skin contrasts conventional products, such as topical anesthetics, that can take anywhere from one minute to sixty minutes to take effect. During this period of numbing, a doctor, nurse, or clinician can perform a medical procedure involving a needle stick (e.g. a shot, vaccine, skin prick, incision, etc.), while minimizing pain to the patient during the medical procedure. Alternatively, the patient can perform a needle stick on himself or herself during this period of numbing, such as a skin prick for daily blood glucose testing for diabetic patients or a needle stick for arthritic patients. In view of the foregoing, the present invention is capable of being used by the patient, as opposed to only a doctor or a nurse, in either a clinical or home setting.

The TENS-like device is preferably a conductive wire mesh 21. When the present invention is in use, the controller 1 generates an electrical signal that is configured with a specific waveform, frequency, and/or pulse rate to electrically stimulate the superficial skin neurons in a particular manner. The controller 1 then sends the electrical signal to the conductive wire mesh 21 so that the conductive wire mesh 21 is able to output the electrical signal onto the patient's skin. The electrical stimulation of the superficial skin neurons places the neurons into a state of fibrillation, which blocks subsequent signals (e.g. pain signals) from reaching the same neurons. Thus, once the superficial skin neurons are in this state of fibrillation, a medical procedure involving a needle stick can be painlessly performed to this target area of the patient's skin. In addition, the conductive wire mesh 21 is integrated into the skin-bracing interface 3, which needs to be electrically conductive, so that the conductive wire mesh 21 is able to output the electrical signal through the skin-bracing interface 3 and onto the patent's skin.

In order for the conductive wire mesh 21 to properly output the electrical signal, the conductive wire mesh 21 needs to comprise a first set of wires 201 and a second set of wires 202. The first set of wires 201 is configured to have a positive electrical potential, while the second set of wires 202 is configured to have a negative electrical potential. In addition, the first set of wires 201 and the second set of wires 202 are positioned perpendicular to each other. This configuration for the first set of wires 201 and the second set of wires 202 is necessary for the conductive wire mesh 21 to properly output the electrical signal onto the patient's skin. The conductive wire mesh 21 and the controller 1 should work in conjunction to produce a preferred electrical signal, which ranges from 20 to 90 volts (V) at approximately 90 Hertz (Hz). An alternative electrical signal could be produced at 60 to 120 Hz. More specifically, the controller 1 comprises a positive terminal 11, which is electrically connected to the first set of wires 201, and further comprises a negative terminal 12, which is electrically connected to the second set of wires 202. A set of leads is used to electrically connect the first set of wires 201 to the positive terminal 11 and to electrically connect the second set of wires 202 to the negative terminal 12. In addition, the first set of wires 201 and the second set of wires 202 are also electrically insulated from each other. This more specific arrangement between the first set of wires 201 and the second set of wires 202 allows the first set of wires 201 to have the positive electrical potential and allows the second set of wires 202 to have the negative electrical potential. In one embodiment, the first set of wires 201 and the second set of wires 202 each include ten wires that are distributed across the conductive wire mesh 21 in their respective direction. Once the conductive wire mesh 21 makes physical contact with the patient's skin, this embodiment allows the patient's skin to be electrically stimulated in 100 places. Moreover, the physical shape of the conductive wire mesh 21 can desirably stimulate the additional sense of pressure, as the conductive wire mesh 21 applies pressure on the patient's skin in unexpected square shapes.

In some embodiments, the waveform of the electrical signal transferred to the conductive wire mesh 21 is a combination of a nerve stimulation waveform and a muscle stimulation waveform (e.g. a ReBuilder waveform). This type of waveform causes the forearm muscles to imitate the action of squeezing a ball several times (a technique that is commonly used in clinics), which utilizes the venous muscle pump to bring more blood into the veins and arteries. Full arteries are necessary in certain medical procedures, such as a needle stick for glucose testing, while full veins are necessary for performing other medical procedures, such as venipuncture blood draws and injections. Moreover, the ability of the waveform to activate the muscles and provide more blood flow into the veins and arteries can further be achieved without the need for a tourniquet. Also in some embodiments, a secondary, additive vibration in the ultrasound range of 20 kHz up to several GHz could be included with the waveform.

As discussed before, the electrical signal transferred from the controller 1 to the conductive wire mesh 21 can further include a specific frequency. In one embodiment, the frequency of the impulses (e.g. TENS impulses) of the electrical signal is about 7.83 Hz. A frequency of impulse in this range provides a comfortable feeling to the patient, while giving the nerve cells time to recover between each impulse. This avoids the nervous system going into a “fight or flight” mode and causing vasoconstriction. However, the intensity and/or duration of the electrical signal can be controlled by the user, which includes, for example, the patient, doctor, nurse, or clinician. Thus, the present invention can allow for patient modulation of at least the electrical stimulation and/or sensation.

The thermoelectric device is preferably a Peltier-effect thermoelectric disk 22 that comprises a cool side 203 and a hot side 204. The Peltier effect allows for cooling of the cool side 203 by providing a current or voltage to the Peltier-effect thermoelectric disk 22 and then converting this current or voltage into a temperature difference felt between the cool side 203 and the hot side 204. The cooling of the cool side 203 is done by applying the current or voltage to the respective positive and negative terminals of the Peltier-effect thermoelectric disk 22. In one embodiment, the Peltier-effect thermoelectric disk 22 operates on a direct current (DC) power source. In a more specific embodiment, the Peltier-effect thermoelectric disk 22 operates on a 12-volt DC power source. Moreover, the skin-bracing interface 3 needs to be thermally conductive, and the cool side 203 needs to be in thermal communication with the skin-bracing interface 3. This allows the Peltier-effect thermoelectric disk 22 to draw heat away from the patient's skin and consequently cool a target area of the patient's skin. The Peltier-effect thermoelectric disk 22 is used to reduce the temperature of the target area of the patient's skin to the same temperature of the cool side 203, which provides a numbing effect to the target area of the patient's skin. This numbing effect, as discussed above, allows for the patient to feel nothing in the target area of the patient's skin for about 30 seconds, such that a medical procedure, such as a needle stick, can be painlessly performed. The rate of cooling obtained by the Peltier-effect thermoelectric disk 22 is controlled manually or automatically for safety, as is the length of cooling time and lowest temperature achieved. In one embodiment, the Peltier-effect thermoelectric disk 22 and the controller 1 are configured with a specific rate of cooling the patient's skin to approximately 30° F. to 38° F. In another embodiment, the temperature of the Peltier-effect thermoelectric disk 22 is controlled at a rate such that the lowest temperature achieved by the cool side 203 is 34° F.

The hot side 204 of the Peltier-effect thermoelectric disk 22 can be in thermal communication with a heat sink 5 in order to keep the Peltier-effect thermoelectric disk 22 from overheating. The heat sink 5 also helps the Peltier-effect thermoelectric disk 22 by transferring heat from the patient's skin to the heat sink 5 during the aforementioned cooling process. The heat sink 5 is preferably made of metal. However, any material that functions as an effective heat sink may be used. In some embodiments, if the at least one neural-overloading source 2 does not include the Peltier-effect thermoelectric disk 22, then a heat sink 5 can be in direct thermal communication with the skin-bracing interface 3, which allows the heat sink 5 to directly draw heat away from the patient's skin. In an alternative embodiment, instead of using the heat sink 5, an internal fan could be used to keep the Peltier-effect thermoelectric disk 22 from overheating.

The vibration device is preferably a piezoelectric vibrating device 23, which vibrates according to a specific electrical signal or waveform that is sent by the controller 1. More specifically, the piezoelectric vibrating device 23 is electrically connected to the controller 1 via respective leads, such that a positive terminal of the piezoelectric vibrating device 23 is connected to the positive terminal 11 of the controller 1, and a negative terminal of the piezoelectric vibrating device 23 is connected to the negative terminal 12 of the controller 1. The piezoelectric vibrating device 23 converts the specific electrical signal or waveform into a physical vibration, which is transferred to the skin-bracing interface 3 because the piezoelectric vibrating device 23 is in percussive communication with the skin-bracing interface 3. Instructions on how to output the physical vibration is preferably delivered to the piezoelectric vibrating device 23 via a specific waveform with a fixed frequency and pulse rate. The physical vibration of the piezoelectric vibrating device 23 is transferred to a target area of the patient's skin that the present invention is placed against, which also provides a numbing or numbness effect by overloading the skin's vibration sensors. This numbing effect, as discussed above, allows for the patient to feel nothing in this area of the skin for a certain amount of time, such that a medical procedure, such as a needle stick, can be painlessly performed. For example, the vibration can be delivered with a waveform having an amplitude of about 4 meters per second squared (m/s²) to about 25 m/s², or more preferably, about 6 m/s² to about 25 m/s², to cause a condition of numbness in the target area of the patient's skin that the present invention is placed against. This amplitude can be measured using an accelerometer. Moreover, the intensity and/or duration of the physical vibration can be controlled by the user, which includes, for example, the patient, doctor, nurse, or clinician. Thus, the present invention can allow for patient modulation of the physical vibration from the piezoelectric vibrating device 23. Also in one embodiment, the physical vibration generated by the piezoelectric vibrating device 23 is coupled with low intensity extracorporeal shock wave therapy (LI-ESWT), which provides intermittent shockwaves to the patient's skin that cause vasodilation resulting in more superficial blood flow that is beneficial during, for example, finger sticks for blood glucose monitoring.

In some embodiments, the present invention further comprises a disposable cap 7. The disposable cap 7, which is similar to, for example, a thimble, is preferably made of a rigid plastic (e.g. polymer) and covers the skin-bracing interface 3. Because the disposable cap 7 covers the skin-bracing interface 3 that may include the conductive wire mesh 21, the disposable cap 7 is preferably made of a thin, film stretchable plastic that is also electrically conductive. For example, the conductivity of the disposable cap 7 may be achieved by applying (i) a thin spray or film of conductive paint with a grounding self-adhesive patch to the disposable cap 7, or (ii) a screen with negative and positive polarity wires made of, for example, silver thread to the disposable cap 7. In addition, the disposable cap 7 may further be prepared to have antibiotic and/or antiviral properties. An alternative to the disposable cap 7 is an embodiment of the present invention where the entire invention is covered by an apron or sheath. Also in some embodiments, the present invention further comprises a plurality of nubs 6 that are used to press against the patient's skin during application of the present invention in order to overload the pressure/mechano-sensors on the skin. The plurality of nubs 6 can be distributed across and connected onto the skin-bracing interface 3. Alternatively, the plurality of nubs 6 can be distributed across and connected onto the disposable cap 7.

In one embodiment, the present invention is about the size of a salt shaker. Thus, the skin cooling device is about 0.75 inches across and about 3 inches tall. In this embodiment, the various components of the present invention are sized to fit within these dimensions. Thus, the conductive wire mesh 21 is a thin wire mesh material, the Peltier-effect thermoelectric disk 22 is about 0.125 inches thick, and the piezoelectric vibrating device 23 is configured to be about the size of a buzzer in a watch. Consequently, the heat sink 5 encompasses the majority of the present invention.

Accordingly, the heat sink 5 can be manufactured to be of the size and shape that accommodates the overall size and shape of the present invention. In another embodiment, the present invention can be configured into an overall shape that a pediatric patient would find pleasing. For example, the present invention could be configured into the shape of an animal or other desirable shapes, such as a puppy, kitten, clown, car, truck, ladybug, happy face, etc. In this embodiment, the skin-bracing interface 3 could embody the puppy's nose. Alternatively, the present invention could be configured to be placed within a hand puppet, such as an animal (e.g. a rabbit) or doll with the nose of the hand puppet being the skin-bracing interface 3.

In some embodiments, the present invention includes a mechanism to control the pressure exerted onto the surface of the patient's skin by the skin-bracing interface 3. For example, the mechanism to control the pressure can be a spring or a spring type housing that separates the various active components of the present invention and presses certain components towards (or away from) the skin-bracing interface 3. This type of spring or spring type housing can ensure that a uniform, safe pressure is being exerted onto the surface of the patient's skin.

In some embodiments, the present invention includes an audible alerting device 8 that emanates an audible sound within the 20 Hz range of human hearing. For example, the audible sound could be about 7 Hz. This audible alerting device 8 can (i) indicate to a user that the present invention is active and (ii) function as a distraction so that the user will not focus on the medical procedure to be performed. These functionalities of the audible alerting device 8 can only be enabled if the audible alerting device 8 is electronically connected to the controller 1.

In some embodiments, the present invention further comprises at least one biofeedback sensor 9 in order to provide useful operational information on any of the at least one neural-overloading source(s) 2 and/or provide useful information on the physical status of the patient's skin. The at least one biofeedback sensor 9 needs to be electronically connected to the controller 1 so that the sensing data can be exchanged between the at least one biofeedback sensor 9 and the controller 1.

In regards to the TENS-like device as a neural-overloading source 2, the at least one biofeedback sensor 9 can include (i) an ohmmeter 91 to measure the electrical resistance of the patient's skin, (ii) a multimeter 92 to measure the initial voltage and/or current of the electrical signal from the TENS-like device, and/or (iii) the same or a different multimeter 92 to monitor and adjust the electrical signal from TENS-like device so that the present invention can maximize the numbing effect for a patient while minimizing the potential discomfort for the patient. The ohmmeter 91 needs to be operatively integrated into the skin-bracing interface 3 so that, while the skin-bracing interface 3 is physically contacting the patient's skin, the ohmmeter 91 is able to measure the electrical resistance across the patient's skin. In addition, the multimeter 92 is operatively coupled to the TENS-like device, which allows the multimeter 92 to measure electrical properties of the output signal generated by the TENS-like device, such as time-dependent current or voltage.

In regards to the thermoelectric device as a neural-overloading source 2, the at least one biofeedback sensor 9 can also include (i) a temperature sensor 93 to measure the patient's initial skin temperature, (ii) the same or a different temperature sensor 93 to measure the skin temperature as the temperature is decreasing, (iii) the same or a different temperature sensor 93 to measure the final skin temperature, and/or (iv) another temperature sensor 94 to interact with the controller 1 in order to deliver the correct amount of voltage and/or current to the thermoelectric device. The temperature sensor 93 is operatively integrated into the skin-bracing interface 3 so that, while the skin-bracing interface 3 is physically contacting the patient's skin, the temperature sensor 93 is able to measure the temperature of the patient's skin. The other temperature sensor 94 is also operatively integrated into the thermoelectric device, which allows the other temperature sensor 94 to measure the temperature of the thermoelectric device. Preferably, a feedback thermistor (e.g. a biofeedback control) is used as the other temperature sensor 94 to monitor the temperature of the patient's skin during the cooling process and to control the time that the thermoelectric device is powered on while touching the patient's skin. By providing this feedback thermistor, the length of the cooling time and the lowest temperature achieved can be automatically adjusted via a biofeedback control mechanism for safety purposes.

In regards to the vibration device as a neural-overloading source 2, the at least one biofeedback sensor 9 can also include a piezoelectric sensing device 95 to monitor and adjust the rate of physical vibration generated by the vibration device and/or the current (i.e. strength) applied thereto in order to maximize the physical vibration of the patient's skin. The piezoelectric sensing device 95 is operatively integrated into the skin-bracing interface 3 so that, while the skin-bracing interface 3 is physically contacting the patient's skin, the piezoelectric sensing device 95 is able to measure the physical oscillation of the patient's skin. For example, underlying fat can diminish or cushion the vibration, while bone or scar tissue, etc., can return a stronger vibration response. The combination of the piezoelectric vibrating device 23 and the piezoelectric sensing device 95 can be used as an elasticity sensor to measure skin elasticity (e.g. firmness) by determining the elastin (i.e. elasticity) and/or collagen fibers (i.e. firmness) of the dermis. Due to aging and other external factors, the elasticity of the skin generally deteriorates over time. The elasticity sensor can, for example, apply a measurement technique in which the vibrator oscillates at a particular frequency and, when applied to the skin, a change in the frequency can reflect the firmness of the skin. For example, the greater the change in the frequency measured by the piezoelectric sensing device 95, the more elastic the skin and the lower the frequency/current that is necessary for vibration.

In addition, the at least one biofeedback sensor 9 can further include a capacitive sensor 96 to measure surface skin moisture. The capacitive sensor 96 is operatively integrated into the skin-bracing interface 3 so that, while the skin-bracing interface 3 is physically contacting the patient's skin, the capacitive sensor 96 is able to measure surface moisture of the patient's skin. The higher the capacitance of the skin, for example, the higher the resulting score and the more hydrated the skin is at the point of measurement. Measuring the surface skin moisture can be beneficial as skin hydration can affect the sensation and/or comfort level of the device. The measured surface skin moisture can also be used as feedback for the functioning of the other modalities (e.g. electrical stimulation, cooling, and/or vibration). In addition, the at least one biofeedback sensor 9 can further include a photometric sensor 97. The photometric sensor 97 can shine a light onto the skin and measure the reflectance back to determine and monitor lipids (e.g. oil) on the skin, which can affect conductivity and thereby, require more voltage/current from, for example, the TENS-like device. Thus, the photometric sensor 97 needs to be oriented towards the normal direction of the skin-bracing interface 3, which allows the photometric sensor 97 to be oriented towards the patient's skin and consequently enables the photometric sensor 97 to measure light reflectance off the patient's skin.

Even through the present invention can utilize all three-separate neural-overloading source(s) 2 (i.e. the conductive wire mesh 21 to provide electrical stimulation to the superficial skin neurons, the Peltier-effect thermoelectric disk 22 to provide the cooling and/or numbing effect, and the piezoelectric vibrating device 23 to provide a numbing effect by overloading the skin's vibration sensors), the present invention can be configured to include one, two, or three of these separate neural-overloading source(s) 2. For example, one embodiment of the present invention is only able to cool the patient's skin and consequently includes only the thermoelectric device. Another embodiment of the present invention is only able to electrically stimulate the patient's skin and consequently includes only the TENS-like device. Another embodiment of the present invention is only able to physically vibrate the patient's skin and consequently includes only the vibration device. Another embodiment of the present invention is only able to cool and electrically simulate the patient's skin and consequently includes only the thermoelectric device and the TENS-like device. Another embodiment of the present invention is only able to cool and physically vibrate the patient's skin and consequently includes only the thermoelectric device and the vibrating device. Another embodiment of the present invention is only able to electrically stimulate and physically vibrate the patient's skin and consequently includes only the TENS-like device and the vibrating device. In addition, other possible neural-overloading source(s) 2 could be incorporated into the present invention, including, for example, pressure and/or movement. Moreover, the functioning of the present invention can be configured to be user-modulated such that the length of time and/or rate of cooling, the electrical stimulation, and/or the vibration rate can be controlled by the user, which includes the patient, doctor, nurse, or clinician. Alternatively, the functioning of the present invention can be configured to be controlled via a computer.

The embodiments described before generally relate to the use of the present invention prior to a medical procedure involving, for example, a needle stick. However, the present invention can further be used by a user (e.g. a patient, doctor, nurse, or clinician) after the medical procedure in order to numb and/or cool the skin if any lingering pain remains after the procedure. In addition, the present invention can further limit any blood loss and/or inflammation accompanying the medical procedure (e.g. needle stick) due to the superficial vasoconstriction of the cooling process.

In terms of industrial applicability, the present invention provides a beneficial way of temporarily numbing the superficial skin neurons rapidly and effectively, in order to allow for medical procedures involving needle sticks to be performed painlessly and without mental distress to the patient. The present invention can function automatically, with the ability of the user to modulate the functioning of the device. Moreover, the present invention is capable of being handled and used by either the patient and/or the clinician.

Although the invention has been explained in relation to its preferred embodiment, it is to be understood that many other possible modifications and variations can be made without departing from the spirit and scope of the invention as hereinafter claimed.

What is claimed is:

1. A skin desensitizing device comprises:
a controller;
at least one neural-overloading source;
a skin-bracing interface;
a power source;
the at least one neural-overloading source being selected from the group consisting of a transcutaneous electrical nerve stimulator (TENS)-like device, a thermoelectric device, a vibration device, and any combination thereof;
the at least one neural-overloading source being electrically connected to the power source through the controller; and
the skin-bracing interface being operatively coupled to the at least one neural-overloading source, wherein the skin-bracing interface is used as a medium between a patient's skin and the at least one neural-overloading source.

2. The skin desensitizing device as claimed in claim 1 comprises:
the TENS-like device being a conductive wire mesh;
the conductive wire mesh comprises a first set of wires and a second set of wires;
the skin-bracing interface being electrically conductive;
the conductive wire mesh being integrated into the skin-bracing interface;
the first set of wires and the second set of wires being positioned perpendicular to each other;
the first set of wires being configured to have a positive electrical potential; and
the second set of wires being configured to have a negative electrical potential.

3. The skin desensitizing device as claimed in claim 2 comprises:
the controller comprises a positive terminal and a negative terminal;
the first set of wires being electrically connected to the positive terminal;
the second set of wires being electrically connected to the negative terminal; and
the first set of wires and the second set of wires being electrically insulated from each other.

4. The skin desensitizing device as claimed in claim 2, wherein the conductive wire mesh and the controller are configured to generate a 20-volt to 90-volt output signal at approximately 90 Hz.

5. The skin desensitizing device as claimed in claim 1 comprises:
the thermoelectric device being a Peltier-effect thermoelectric disk;
the Peltier-effect thermoelectric disk comprises a cool side and a hot side;
the skin-bracing interface being thermally conductive; and
the cool side being in thermal communication with the skin-bracing interface.

6. The skin desensitizing device as claimed in claim 5, wherein the Peltier-effect thermoelectric disk and the controller are configured with a specific rate of cooling the patient's skin to approximately 30° F. to 38° F.

7. The skin desensitizing device as claimed in claim 5 comprises:
a heat sink; and
the heat sink being in thermal communication with the hot side.

8. The skin desensitizing device as claimed in claim 1 comprises:
the vibration device being a piezoelectric vibrating device; and
the piezoelectric vibrating device being in percussive communication with the skin-bracing interface.

9. The skin desensitizing device as claimed in claim 8, wherein the piezoelectric device and the controller are configured to produce a vibration at amplitude of about 4 m/s² to about 25 m/s².

10. The skin desensitizing device as claimed in claim 1 comprises:
a heat sink; and
the heat sink being in thermal communication with the skin-bracing interface/

11. The skin desensitizing device as claimed in claim 1 comprises:
a plurality of nubs;
the plurality of nubs being distributed across the skin-bracing interface; and
the plurality of nubs being connected onto the skin-bracing interface.

12. The skin desensitizing device as claimed in claim 1 comprises:
a disposable cap;
the disposable cap being electrically conductive; and
the disposable cap being detachably attached onto the skin-bracing interface.

13. The skin desensitizing device as claimed in claim 1 comprises:
an audible alerting device; and
the audible alerting device being electronically connected to the controller.

14. The skin desensitizing device as claimed in claim 1 comprises:
at least one biofeedback sensor; and
the at least one biofeedback sensor being electronically connected to the controller.

15. The skin desensitizing device as claimed in claim 14 comprises:
the at least one biofeedback sensor comprises an ohmmeter; and
the ohmmeter being operatively integrated into the skin-bracing interface,
wherein the ohmmeter is used to measure electrical resistance across the patient's skin.

16. The skin desensitizing device as claimed in claim 14 comprises:
the at least one biofeedback sensor comprises a multimeter; and
the multimeter being operatively coupled to the TENS-like device,
wherein the multimeter is used to measure electrical properties of an output signal generated by the TENS-like device.

17. The skin desensitizing device as claimed in claim 14 comprises:
the at least one biofeedback sensor comprises a temperature sensor; and
the temperature sensor being operatively integrated into the skin-bracing interface, wherein the temperature sensor is used to measure temperature of the patient's skin.

18. The skin desensitizing device as claimed in claim 14 comprises:
the at least one biofeedback sensor comprises a temperature sensor; and
the temperature sensor being operatively integrated into the thermoelectric device, wherein the temperature sensor is used to measure temperature of the thermoelectric device.

19. The skin desensitizing device as claimed in claim 14 comprises:
the at least one biofeedback sensor comprises a piezoelectric sensing device; and
the piezoelectric sensing device being operatively integrated into the skin-bracing interface, wherein the piezoelectric sensing device is used to measure physical oscillation of the patient's skin,

20. The skin desensitizing device as claimed in claim 14 comprises:
the at least one biofeedback sensor comprises a capacitive sensor; and
the capacitive sensor being operatively integrated into the skin-bracing interface, wherein the capacitive sensor is used to measure surface moisture of the patient's skin.

21. The skin desensitizing device as claimed in claim 14 comprises:
the at least one biofeedback sensor comprises a photometric sensor; and
the photometric sensor being oriented towards a normal direction of the skin-bracing interface, wherein the photometric sensor is used to measure light reflectance off the patient's skin.
